(19) United States
(12) Patent Application Publication
(10) Pub. No.: US 2024/0316129 A1
(43) Pub. Date: Sep. 26, 2024

(54) Bacteria and Herbal Extract Nutraceutical Blends for Lung Health Maintenance

(71) Applicants: The UAB Research Foundation, Birmingham, AL (US); RESBIOTIC NUTRITION, INC., Birmingham, AL (US)

(72) Inventors: Charitharth Vivek LAL, Vestavia Hills, AL (US); Nancy Wenger, Birmingham, AL (US)

(21) Appl. No.: 18/570,867

(22) PCT Filed: Jun. 16, 2022
(86) PCT No.: PCT/US2022/072975

§ 371 Date: Dec. 15, 2023

Related U.S. Application Data
(60) Provisional application No. 63/211,887, filed on Jun. 17, 2021.

(51) Int. Cl. A61K 35/747; A61K 9/00; A61K 36/19; A61K 36/53; A61K 36/9066

(52) U.S. Cl. CPC A61K 35/747; A61K 9/0053; A61K 36/19; A61K 36/53; A61K 36/9066

(57) ABSTRACT

A combination of herbal extracts and a probiotic supplement and formulated as a dietary supplement helps mitigate symptoms of bronchopulmonary disease in conjunction with the anti-inflammatory mechanisms of the probiotics. Provided are embodiments of a probiotic nutraceutical composition that delivers 3 strains of Lactobacillus spp. to the gut lining of human subjects to reduce neutrophilic inflammation as a result of or causative agent of bronchopulmonary disease. In addition to the three Lactobacillus spp. strains, the compositions can include herbal extracts from holy basil, turmeric, and vasaka for respiratory relief.

### CROSS-REFERENCE TO RELATED APPLICATIONS

This application claims priority to U.S. Provisional Patent Application Ser. No. 63/211,887 entitled “PROBIOTIC BACTERIA MIX AND HERB-BASED NUTRACEUTICALS FOR LUNG HEALTH MAINTENANCE” filed on Jun. 17, 2021, the entirety of which is hereby incorporated by reference.

### TECHNICAL FIELD

The present disclosure encompasses embodiments of a probiotic bacteria and herb-based nutraceutical composition for the maintenance of lung health.

### BACKGROUND

The microbiomes of the lung and gut have been well-established, and species continue to be characterized through microbiome analysis based on sequencing of 16S ribosomal RNA genes (Rogers et al., (2015) Thorax 70:74-81; Segata et al., (2012) Nat. Methods 9:811-814). Dysbiosis has shown to be a contributing factor in asthma (Hilty et al., (2010) PLoS One 5:e8578; Marri et al. 2013), cystic fibrosis (Zhao et al., 2012; Coburn et al., 2015), Bronchopulmonary Dysplasia (Lal & Ambalavanan (2017) Early Hum. Dev. 105:35-39), and Chronic Obstructive Pulmonary Disease (Bowerman et al., (2020) Nat. Commun. 11:5886; Huang et al., (2010) OMICS 14:9-59).

A downstream effect of dysbiosis, whether in the gut or the lungs, is neutrophilic inflammation. Upon an insult to the airway epithelium, collagen is exposed and cleaved by Matrix Metallopeptidase 9 (MMP-9) (Gaggar et al., (2008) J. Immunol. 180):5662-5669; Malik et al., (2007) J. Immunol. 178:1013-1020). MMP-9 and prolyl endopeptidase (PE) cleave the collagen fragments to liberate the acetylated form (Ac-PGP) of the tripeptide Proline-Glycine-Proline (PGP) (Lin et al., (2008) Am. J. Pathol. 173:144-153; Malik et al., (2007) J. Immunol. 178:1013-1020). Ac-PGP binds CXCR2 receptors to trigger neutrophilic inflammation (Weathington et al., (2006) Nat. Med. 12:317-323).

Accordingly, the gut microbiome can impact distant mucosal sites via modulation of the mucosal immune response as well as having induced indirect effects by bacterial byproducts and metabolites (Russell et al., (2012) EMBO Rep. 13:440-447; Anand & Mande (2018) Front Microbiol. 9:2147). Therefore, probiotics can be taken enterally and still impact lung health through the gut-lung axis (Enaud et al., (2020) Front Cell Infect. Microbiol. 10:9).

### SUMMARY

A combination of herbal extracts and a probiotic supplement formulated as a dietary supplement helps mitigate symptoms of bronchopulmonary disease in conjunction with the anti-inflammatory mechanisms of the probiotics. The disclosure encompasses embodiments of a food product, dietary supplement, medical food, or nutraceutical, wherein the food product, dietary supplement, medical food, or nutraceutical can comprise at least viable one probiotic bacterial strain selected from Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, the food product, dietary supplement, medical food, or nutraceutical can comprise a mix of two or three of the probiotic bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, the food product, dietary supplement, medical food, or nutraceutical can comprise a mix of the bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, the food product, dietary supplement, medical food, or nutraceutical can consist of a mix of the bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, at least one Lactobacillus species can be heat-killed.

In some embodiments of this aspect of the disclosure, the percent weights (% wt) of the Lactobacillus plantarum RSB11.RTM, Lactobacillus acidophilus RSB12.RTM, and Lactobacillus rhamnosus RSB13.RTM in the bacterial mix can be, respectively, for RSB11.RTM: 1%-30%; 5%-30%; 5%-20%; 5%-15%; 5%-10%; 10%-30%; 10%-20%; 10%-15%; 15%-30%; 20%-30%, for RSB12.RTM: 1%-30%; 5%-30%; 5%-20%; 5%-15%; 5%-10%; 10%-30%; 10%-20%; 10%-15%; 15%-30%; 20%-30%, and for RSB13.RTM: 1%-30%; 5%-30%; 5%-20%; 5%-15%; 5%-10%; 10%-30%; 10%-20%; 10%-15%; 15%-30%; 20%-30%, with the total percent weights adjusted to 100% wt with at least one excipient, and wherein the ratio of bacteria mix:excipient can be about 10:about 1, about 7:about 1, about 5:about 1, about 4:about 1, about 3:about 1, about 2:about 1, about 1:about 1, about 1:about 2, about 1:about 3, about 1:about 4, about 1:about5, about 1:about7, about 1:about 10.

In some embodiments of this aspect of the disclosure, the percent weights (% wt) of the herbs, or extracts thereof in the herbal mix can be, respectively, turmeric extract: 1%-30%; 5%-30%; 5%-20%; 5%-15%; 5%-10%; 10%-30%; 10%-20%; 10%-15%; 15%-30%; 20%-30%, holy basil extract: 1%-30%; 5%-30%; 5%-20%; 5%-15%; 5%-10%; 10%-30%; 10%-20%; 10%-15%; 15%-30%; 20%-30%, and for vasaka extract: 1%-30%; 5%-30%; 5%-20%; 5%-15%; 5%-10%; 10%-30%; 10%-20%; 10%-15%; 15%-30%; 20%-30% with the total percent weights adjusted to 100% wt with at least one excipient.

In some embodiments of this aspect of the disclosure, the food product, dietary supplement, medical food, or nutraceutical the bacterial mix can consist of Lactobacillus plantarum RSB11.RTM, Lactobacillus acidophilus RSB12.RTM, and Lactobacillus rhamnosus RSB13.RTM, and an excipient consisting of magnesium stearate; silica, and MCC and the herbal mix can consist of turmeric extract, holy basil extract, and vasaka extract.

In some embodiments of this aspect of the disclosure, the food product, dietary supplement, medical food, or nutraceutical can be formulated for oral consumption.

In some embodiments of this aspect of the disclosure, the food product, dietary supplement, medical food, or nutraceutical can further comprise a nutritionally acceptable carrier, nutritionally acceptable diluent, nutritionally acceptable excipient, nutritionally acceptable adjuvant or nutritionally active ingredient in addition to the bacteria and the at least one herb, or extract thereof.

In some embodiments of this aspect of the disclosure, the food product, dietary supplement, medical food, or nutraceutical can be an orally ingestible form selected from the group consisting of a capsule, microcapsule, tablet, granule, powder, troche, pill, suspension, and syrup.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates a model for the influence of dysbiosis on neutrophilic inflammation by upregulating the MMP-9 pathway in human bronchial epithelial cells (NHBE).

FIG. 2 schematically illustrates a model for the influence of dysbiosis on systemic neutrophilic inflammation by upregulating systemic MMP-9 in human intestinal epithelial cells (IEC).

FIG. 3 schematically illustrates a model for the influence of resB on decreasing dysbiosis-triggered neutrophilic inflammation via the MMP-9 pathway in NHBE.

FIG. 4 schematically illustrates a model for the influence of resB on decreasing systemic dysbiosis-mediated neutrophilic inflammation via the MMP-9 pathway in IEC.

FIG. 5 illustrates that MMP-9 levels increase in NHBE cells with the addition of E. coli in increasing concentrations.

FIGS. 6A and 6B illustrate that Proteobacteria (E. coli) leads to an MMP-9 surge in an in vitro (NHBE) dysbiosis model of chronic lung disease.

FIG. 6A illustrates that E. coli increases N-Acetyl Proline-Glycine-Proline (Ac-PGP) concentration isolated from NHBE cell supernatant compared to controls (*P<0.05). Noxious stimuli amplify the effects of E. coli exposure compared to controls (*P<0.05).

FIG. 6B illustrates that E. coli-exposed NHBE cells show increased MMP-9 mRNA levels in supernatant compared to controls (*P<0.05).

FIGS. 7A-7E illustrate that Proteobacteria (E. coli) lead to an MMP-9 pathway surge in an in vivo (mouse) dysbiosis model of chronic lung disease.

FIG. 7A illustrates that LPS-treated C57BL/6 mouse pups show alveolar hypoplasia and simplification in lung tissue.

FIG. 7B illustrates that radial alveolar counts (RAC) were significantly decreased in LPS-treated mice compared to controls (*P<0.05).

FIG. 7C illustrates that myeloperoxidase (MPO) levels were significantly increased in bronchoalveolar lavage fluid (BALF) of LPS-treated mice compared to controls (*P<0.05).

FIG. 7D illustrates that neutrophil elastase (NE) levels were significantly increased in BALF of LPS-treated mice compared to controls (*P<0.05).

FIG. 7E illustrates that Ac-PGP levels were significantly increased in BALF of LPS-treated mice compared to controls (*P<0.05) as measured by HPLC.

FIGS. 8A-8D illustrates that gain of function of Ac-PGP causes chronic lung disease in vivo.

FIG. 8A illustrates that Ac-PGP-treated mice show decreased RAC counts in normoxia and under noxious stimuli (*P<0.05).

FIG. 8B illustrates that MPO concentration increased in Ac-PGP-treated mice in normoxia and under noxious stimuli as measured by ELISA (*P<0.05).

FIG. 8C illustrates that NE concentrations in BALF increased in Ac-PGP-treated mice in normoxia and under noxious stimuli (*P<0.05).

FIG. 8D illustrates that right ventricular hypertrophy (RVH) increased in Ac-PGP-treated mice in normoxia and under noxious stimuli (*P<0.05).

FIG. 9A-9D illustrates the loss of function of Ac-PGP by treatment with the Ac-PGP inhibitor arginine-threonine-arginine (RTR) is protective against chronic lung disease in vivo.

FIG. 9A illustrates that newborn wild type C57BL/6 mice were randomized to treatment with RTR, LPS, and RTR+LPS as well as normoxia and noxious stimuli.

FIG. 9B illustrates that under noxious stimuli, LPS+RTR treated mice showed significantly higher RAC compared to mice treated with LPS alone (*P<0.05).

FIG. 9C illustrates that under noxious stimuli, LPS+RTR treated mice showed significantly lower MPO concentrations than mice treated with LPS alone (*P<0.05).

FIG. 9D illustrates that under noxious stimuli, LPS+RTR treated mice showed significantly less severe RVH than mice treated with LPS alone (*P<0.05).

FIG. 10 illustrates that resB blend #1 of the disclosure reverses the MMP-9 cascade in vitro (NHBE) under increasing concentrations of E. coli.

FIG. 11 illustrates that herbal extracts comprising holy basil, turmeric, and vasaka as well as Vitamin D do not have an inhibitory effect on the culture growth of probiotic strains RSB11, RSB12, or RSB13.

FIG. 12 illustrates that herbal extracts comprising vasaka, turmeric, and organic holy basil reduce MMP-9 in NHBE treated with E. coli.

FIG. 13 illustrates that herbal extracts comprising vasaka, turmeric, and organic holy basil in combination with resB blend #1 reduce MMP-9 in NHBE exposed to E. coli.

FIG. 14 illustrates that the micronutrient Vitamin D in combination with resB blend #1 reduces MMP-9 in NHBE exposed to E. coli.

FIG. 15 illustrates that the micronutrients Vitamin D and prebiotic fiber inulin in combination with resB blend #1 reduce MMP-9 in NHBE exposed to E. coli.

FIGS. 16A-B illustrate that treatment with resB improves lung structure in an in vivo murine model of LPS exposure.

FIG. 16A shows histology slides of mouse lung tissue.

FIG. 16B shows that mean linear intercept (MLI) in mice treated with resB are lower than in mice only exposed to LPS (*P<0.05).

FIGS. 17A-17C illustrate that resB (herbal extracts+resB blend #1) reduces neutrophilic inflammation in the lungs in vivo.

FIG. 17A illustrates that mice treated with LPS+resB exhibit a significantly lower BAL pellet cell count compared to mice treated with saline+LPS (*P<0.05).

FIG. 17B illustrates that mice treated with LPS+resB exhibit a significantly lower BAL neutrophil count compared to mice treated with saline+LPS (*P<0.05).

FIG. 17C illustrates that mice treated with LPS+resB exhibit significantly lower BAL MPO levels compared to mice treated with saline+LPS (*P<0.05).

FIGS. 18A-18B illustrate that resB reduces activity of the MMP-9 pathway in vivo.

FIG. 18A illustrates that resB decreases MMP-9 mRNA levels in mouse lung tissue (*P<0.05, ****P<0.0001).

FIG. 18B illustrates that resB decreases MMP-9 protein levels in the BAL (*P<0.05, ****P<0.0001).

FIGS. 19A-19B illustrate that resB reduces pro-inflammatory marker C-reactive protein in vivo.

FIG. 19A illustrates that resB decreases C-reactive protein (CRP) levels in the BAL (****P<0.0001).

FIG. 19B illustrates that resB decreases C-reactive protein (CRP) levels in the serum (**P<0.01, ****P<0.0001).

FIG. 20 illustrates the consort flow chart for a one-month, open-label, randomized clinical trial of healthy and asthmatic non-smokers and smokers treated with resB.

FIG. 21 is a frequency table of participant demographic data collected at baseline in the safety population.

FIG. 22 is a frequency table for adverse events and severe adverse events by health status and MEDRA SOC in the safety population.

FIG. 23 illustrates improvement in lung function as measured by FEV1 in the asthmatic population from Baseline to Week 4 (*P<0.05).

FIG. 24 illustrates increases in serum SCFA levels in asthmatic smokers and non-smokers who took resB for one month (*P<0.05).

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Embodiments of the present disclosure will employ, unless otherwise indicated, techniques of medicine, biochemistry, molecular biology, pharmacology, and the like, which are within the skill of the art. Such techniques are explained fully in the literature.

The examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It is to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure, allowing for the presence of more than that which is recited so long as basic or novel characteristics of that which is recited is not changed by the presence of more than that which is recited, but excludes prior art embodiments.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include 1%, +2%, 3%, 4%, 5%, 6%, 7%, 8%, 9%, or +10%, or more of the numerical value(s) being modified.

### Definitions

In describing and claiming the disclosed subject matter, the following terminology will be used in accordance with the definitions set forth below.

The term “bacterial population” as used herein refers to a homogeneous population of bacteria consisting of a single strain of a bacterial species or to a heterogeneous population of bacteria consisting of at least two distinguishable strains of a single bacterial species or of a plurality of species.

The term “bronchopulmonary disease” as used herein refers to a disease of the respiratory system, including the lungs and bronchial tree. Pulmonary diseases include, but are not limited to, cystic fibrosis, Chronic Obstructive Pulmonary Disease (COPD), emphysema, asthma, sarcoidosis, chronic bronchitis, bronchopulmonary dysplasia, pulmonary fibrosis, pneumonia, and adult respiratory distress syndrome. The term “respiratory diseases”, as used herein, means diseases or conditions related to the respiratory system. Examples include, but not limited to, airway inflammation, allergy(ies), asthma, impeded respiration, cystic fibrosis (CF), allergic rhinitis (AR), acute respiratory distress syndrome (ARDS), pulmonary hypertension, lung inflammation, bronchitis, airway obstruction, bronchoconstriction, microbial infection, and viral infection such as, but not limited to, severe acute respiratory syndrome (SARS) or severe acute respiratory syndrome coronavirus 2 (COVID).

The term “composition” as used herein refers to a product comprising the specified ingredients in the specified amounts, as well as any product which results, directly or indirectly, from combination of the specified ingredients in the specified amounts.

The term “food product” as used herein is intended to encompass any consumable matter of either plant or animal origin or of synthetic sources that contain a body of nutrients such as a carbohydrate, protein, fat vitamin, mineral, etc. The product is intended for the consumption by humans or by animals.

The term “formulation” as used herein refers to a composition that may be a stock solution of the components, or a composition, preferably including a dilutant such as water or other pharmaceutically acceptable carrier, that may be available for distribution including to a patient or physician.

The terms “freeze-dried” and “lyophilized” as used herein refer to a preparation of bacterial cells that have been initially frozen and the water content removed by vacuum.

The terms “inactivated probiotic” or “inactivated micro-organism” as used herein mean that the metabolic activity or reproductive ability of the organism has been reduced or destroyed. The inactivated organisms do, however, still retain, at the cellular level, at least a portion of their biological glycol-protein and DNA/RNA structure. As used herein, the term “inactivated” is synonymous with “non-viable”.

The term “Lactobacillus” as used herein refers to a genus of Gram-positive, facultative anaerobic or microaerophilic, rod-shaped, non-spore-forming bacteria. They are a major part of the lactic acid bacteria group (i.e. they convert sugars to lactic acid). Preferred embodiments of the disclosure include at least one strain of at least one of the Lactobacillus species Lactobacillus plantarum, Lactobacillus acidophilus and Lactobacillus rhamnosus.

The term “nutraceutical” as used herein, refers to a food stuff (as a fortified food or a dietary supplement) that provides health benefits.

The term “probiotic” as used herein refers to a microorganism which, when administered in adequate amounts, can confer a health benefit to the host. A probiotic microorganism must fulfil several requirements related to lack of toxicity, viability, adhesion, and beneficial effects. These probiotic features are strain-dependent, even among bacteria of the same species. Therefore, it is important to find those strains that have a better performance in all probiotic requirements.

The term “reducing” means to diminish in extent, amount, or degree.

### Abbreviations

Bronchopulmonary dysplasia, BPD; chronic obstructive pulmonary diseases, COPD; acute exacerbations of chronic obstructive pulmonary diseases, AECOPD; cystic fibrosis, CF; acute respiratory distress syndrome, ARDS; severe acute respiratory syndrome, SARS; severe acute respiratory syndrome coronavirus 2, COVID; Proline-Glycine-Proline, PGP; N-acetyl-Proline-Glycine-Proline, Ac-PGP; Human Bronchial Epithelial Cells, NHBE; human intestinal epithelial cells, IEC; bronchoalveolar lavage fluid, BALF; Arginine-Threonine-Arginine, RTR; myeloperoxidase, MPO; bronchoalveolar lavage fluid, BAL; lipopolysaccharide, LPS

### DESCRIPTION

The present disclosure encompasses embodiments of nutraceutical compositions that can deliver probiotic strains of Lactobacillus spp. to a human or animal subject. Combined with dietary supplements to the epithelial lining of the intestinal tract of recipient subjects. Ingestion of such compositions can lead to relief of inflammation and other pathological conditions of pulmonary disease and can provide benefits to the health of the alimentary canal also. The probiotic compositions, therefore, of the disclosure can comprise at least one strain of Lactobacillus as a live organism capable of proliferation in the subject patient but also can include a non-living strain such as, but not limited to, a heat-killed non-proliferative strain. Some advantageous compositions of the disclosure comprise a combination of three Lactobacillus spp. species of which at least one species is viable. It is further contemplated that the compositions of the disclosure may comprise a plurality of distinguishable strains of the same Lactobacillus species or a plurality of species, each of which may be proliferative, or a combination of proliferative and non-proliferative strains. In some embodiments of the compositions of the disclosure, additional bacterial species or strains thereof may be included with the Lactobacillus spp.

Advantageously, it has been found beneficial to provide a composition that comprises three Lactobacillus species. Most advantageous is a mix of the species Lactobacillus plantarum, Lactobacillus acidophilus, and Lactobacillus rhamnosus of which at least one species is viable in the gut of the recipient subject. In particular, the strains Lactobacillus plantarum RSB11.RTM, Lactobacillus acidophilus RSB12.RTM, and Lactobacillus rhamnosus RSB13.RTM have been found to advantageous for use in the dietary supplements of the disclosure. With respect to all references to “Lactobacillus plantarum RSB11.RTM, Lactobacillus acidophilus RSB12.RTM, and Lactobacillus rhamnosus RSB13.RTM” herein, pursuant to the requirements of the Budapest Treaty, live cultures of the strains designated as Lactobacillus plantarum RSB11.RTM, Lactobacillus acidophilus RSB12.RTM, and Lactobacillus rhamnosus RSB13.RTM were deposited on Nov. 19, 2021 with the American Type Culture Collection Patent Depository and received the deposit numbers PTA-127171 (Lactobacillus plantarum RSB11.RTM), PTA-127172 (Lactobacillus acidophilus RSB12.RTM), and PTA-127173 (Lactobacillus rhamnosus RSB13.RTM), respectively.

In addition to the bacterial strains, the compositions of the disclosure further comprise a herbal mix that in conjunction with probiotic bacteria can provide detectable relief from symptoms of respiratory distress. It has been found advantageous to include in the compositions of the disclosure at least one, two, and most advantageously, all three herbs of the group turmeric, organic holy basil, and vasaka, or extracts thereof.

The compositions of the disclosure, while comprising one or more viable Lactobacillus plantarum RSB11.RTM, Lactobacillus acidophilus RSB12.RTM, and Lactobacillus rhamnosus RSB13.RTM bacterial strains together with at least one herbal extract, can further include one or more nutraceutical or dietary supplements well known to those of skill in the art of nutraceutical or dietary supplements and which are intending to contribute to the health of the recipient animal or human. Thus, the terms “nutritional supplement” or “nutraceutical” as used herein includes, but is not limited to, vitamins, micronutrients, minerals, fiber, fatty acids, and amino acids. Suitable nutritional supplements for use with the compositions of the present disclosure include, but are not limited to: vitamins such as ascorbic acid (vitamin C), vitamins D, E, A, E, K, thiamine (B1), riboflavin (B2), niacin (B3), pantothenic acid (B5), B6, B7, B9, and B12; minerals such as sulfur, potassium, chlorine, sodium, calcium, phosphorus, magnesium, iron, zinc, manganese, copper, iodine, chromium, molybdenum, selenium and cobalt; proteins and amino acids, fiber such as inulin and the like.

In addition, the disclosure encompasses embodiments of a method of reducing symptoms associated with chronic pulmonary disease by administering to a patient in need a volume of a probiotic composition as herein described. Bronchopulmonary dysplasia (BPD), Cystic fibrosis (CF), Chronic Obstructive Pulmonary Disease (COPD) and acute respiratory distress syndrome (ARDS) are chronic lung diseases spanning from infancy to old age. Together these respiratory diseases affect more than 25 million Americans each year. The common problem in all these respiratory conditions is inflammation initiated by injury to the lung by neutrophil influx, resulting in a persistent inflammatory process leading to extracellular matrix (ECM) remodeling followed by impaired lung structure and function.

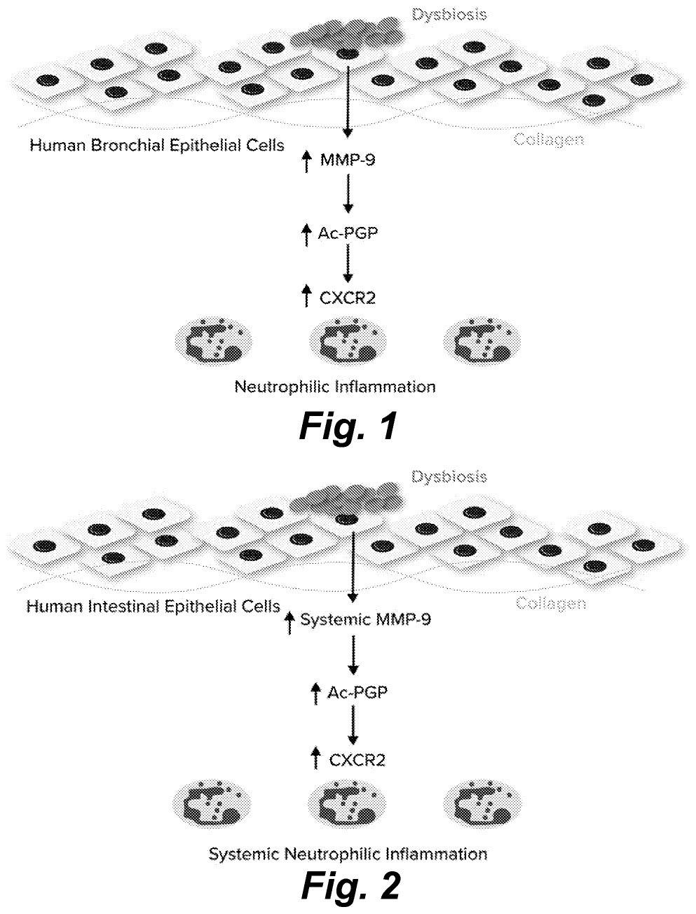

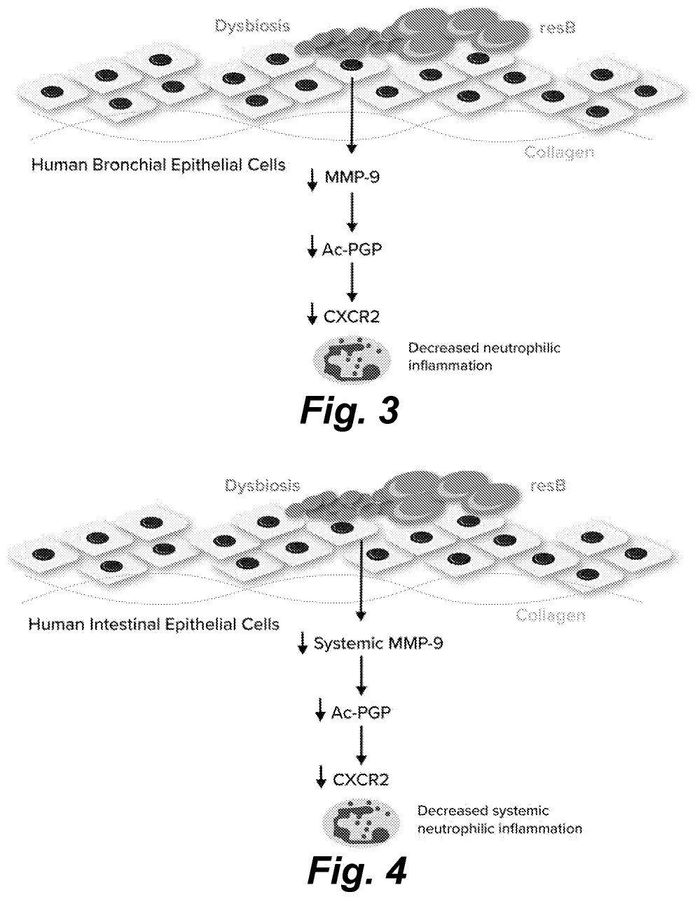

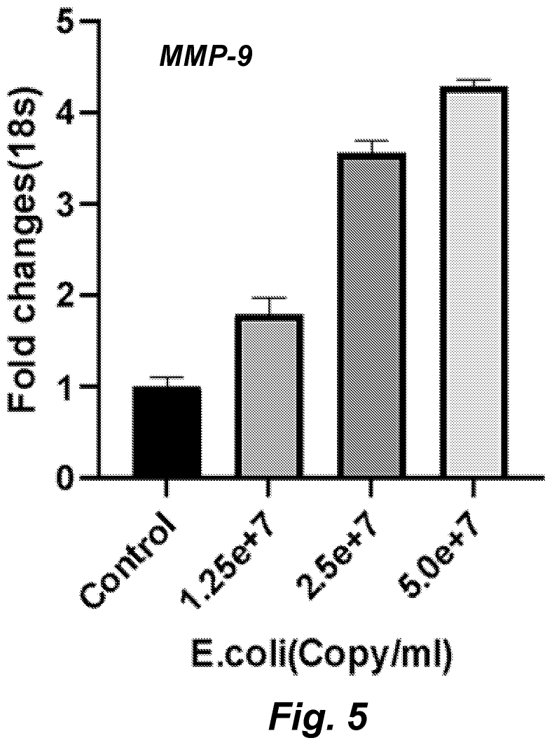

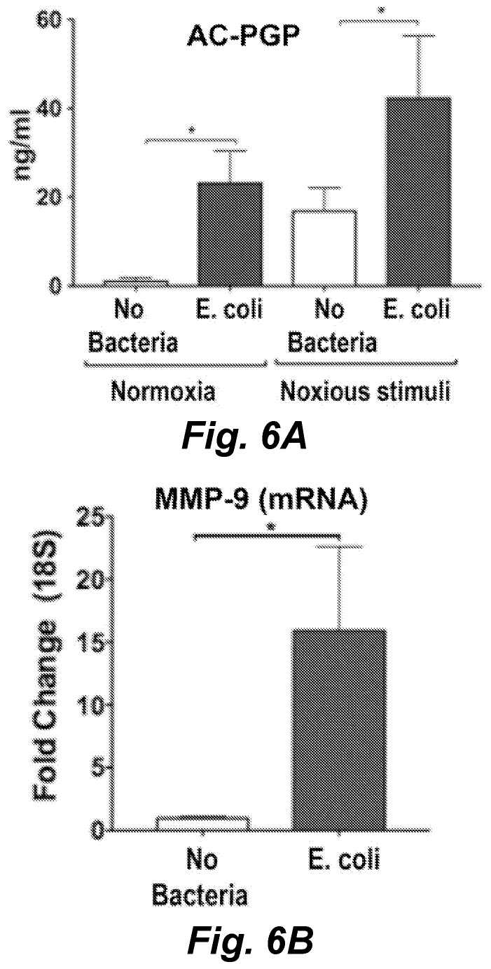

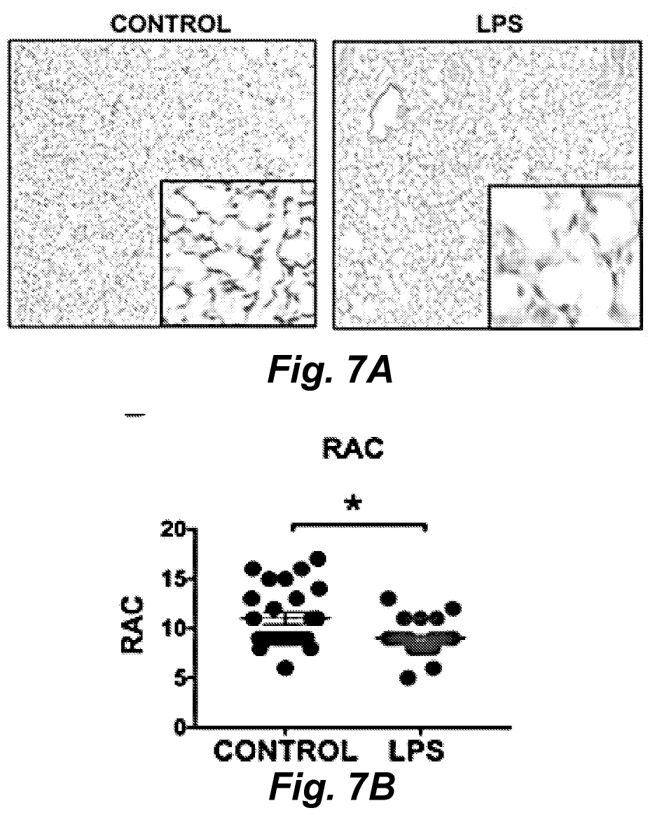

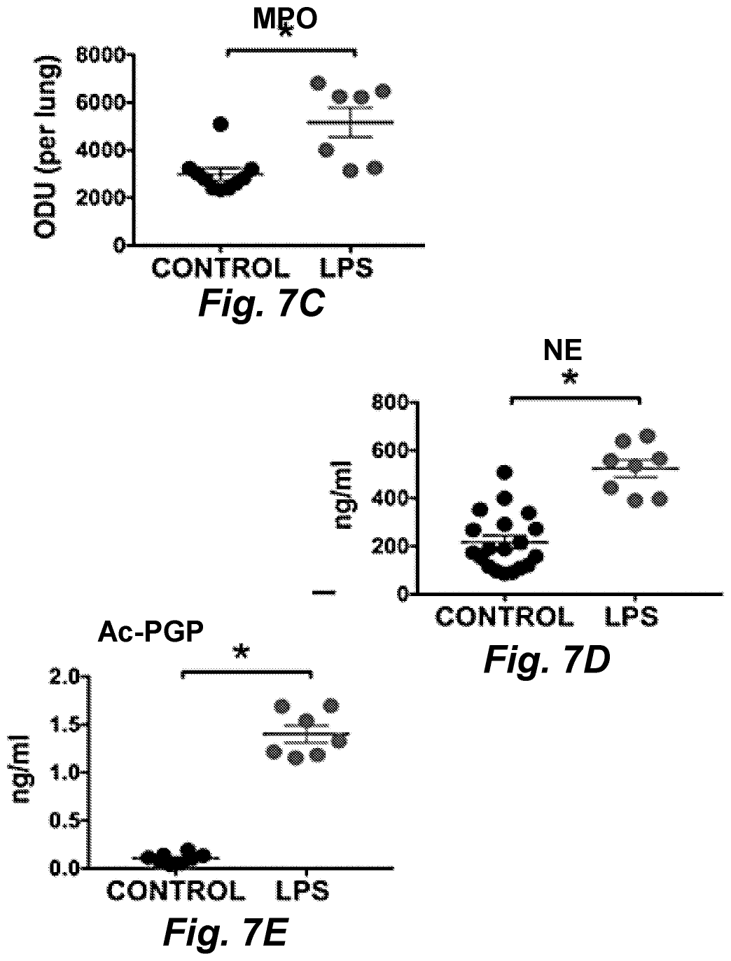

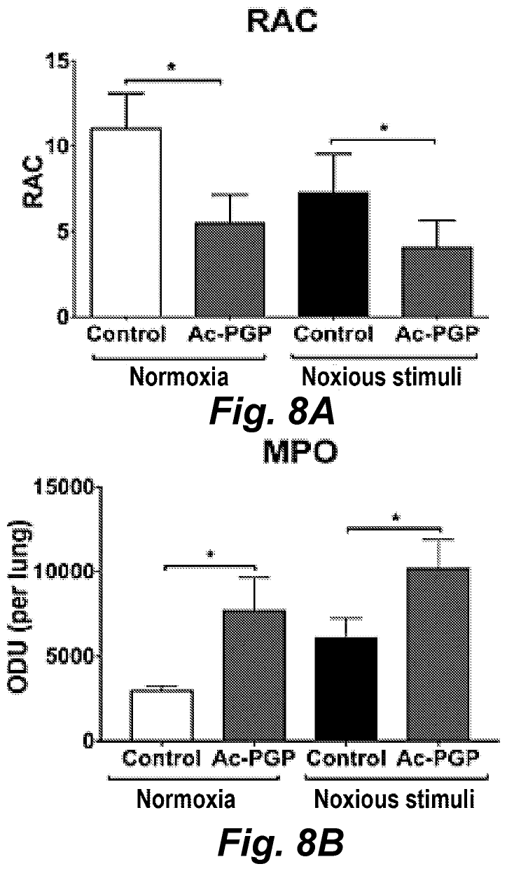

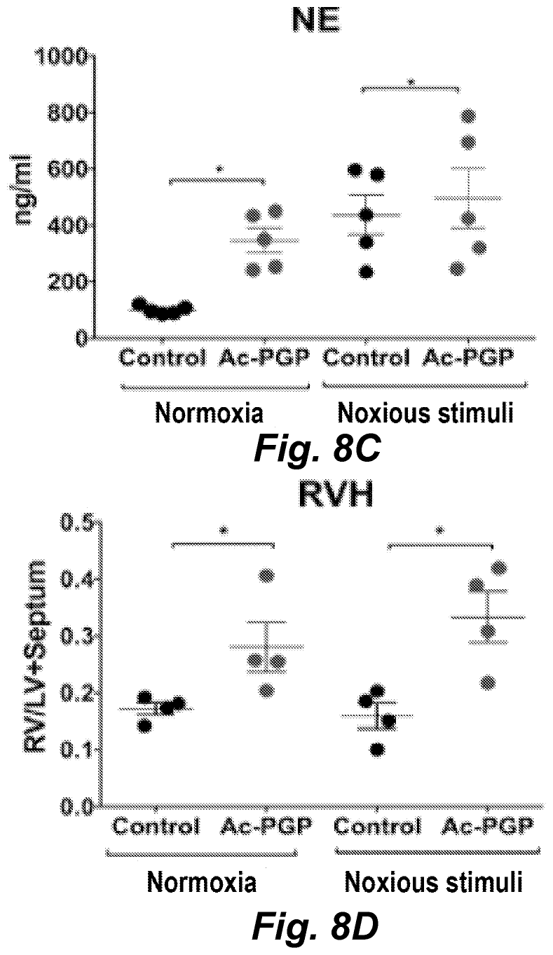

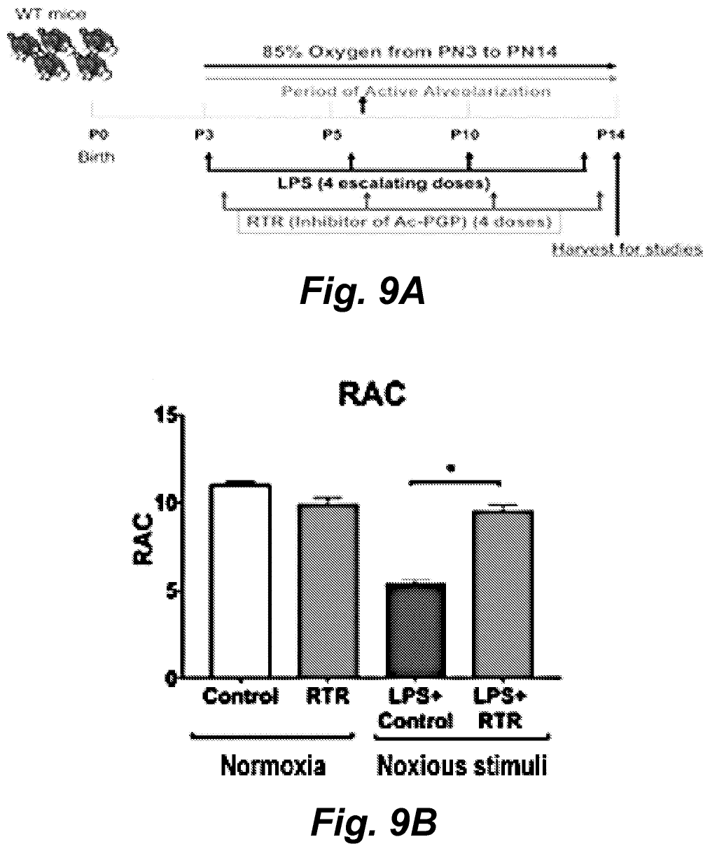

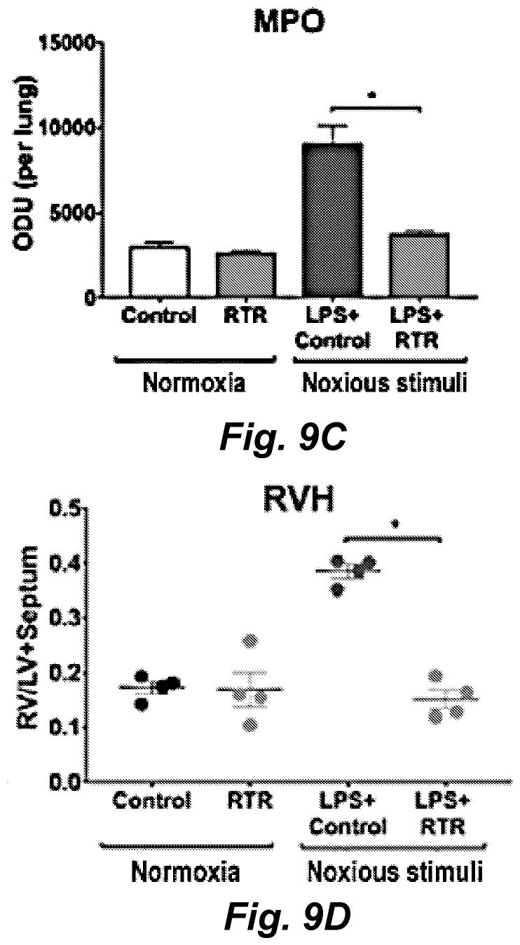

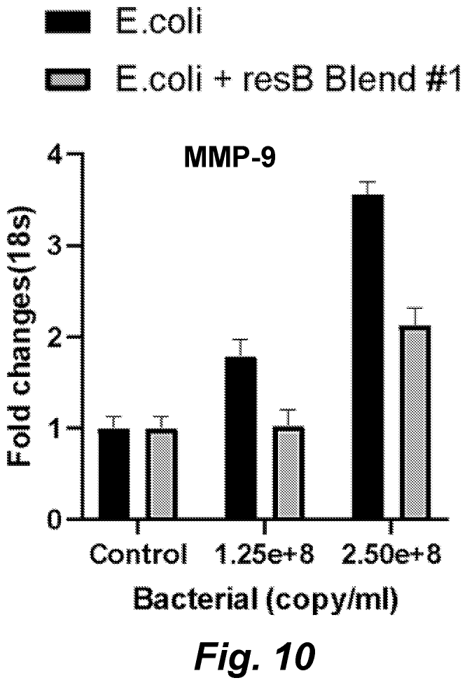

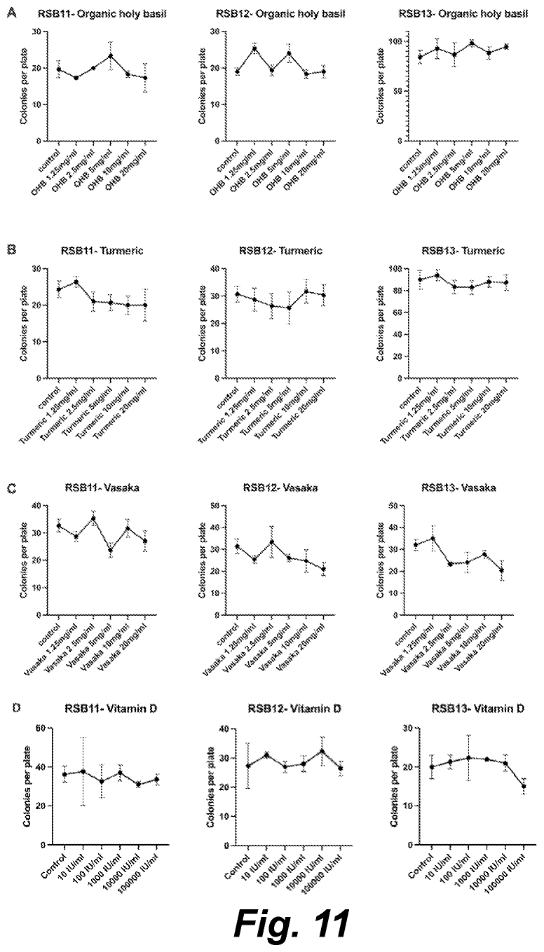

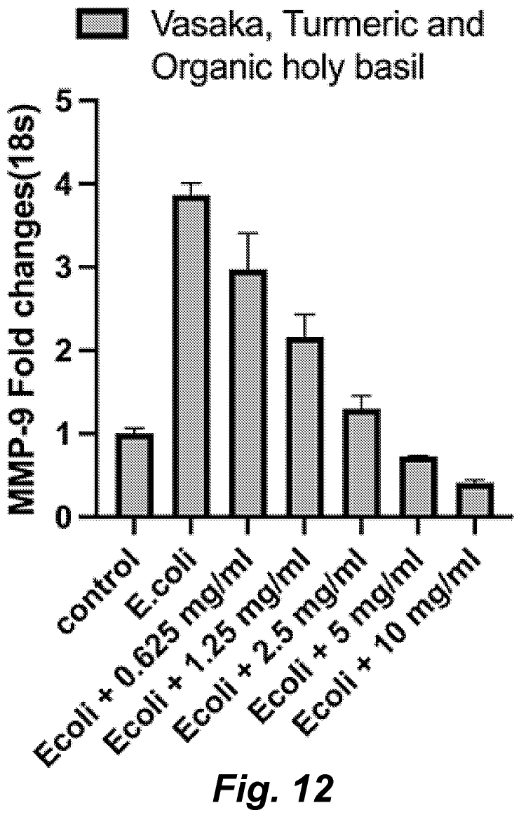

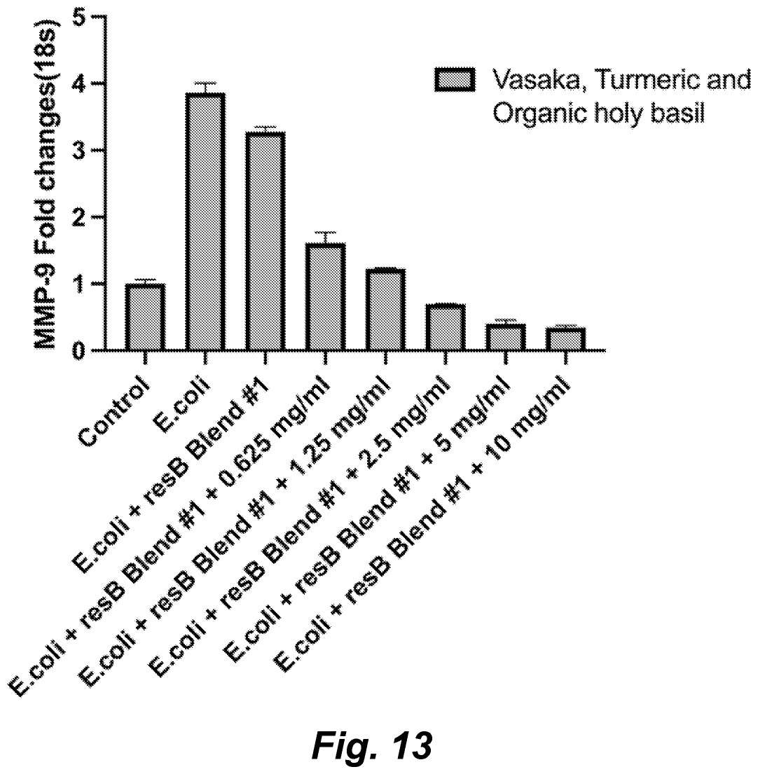

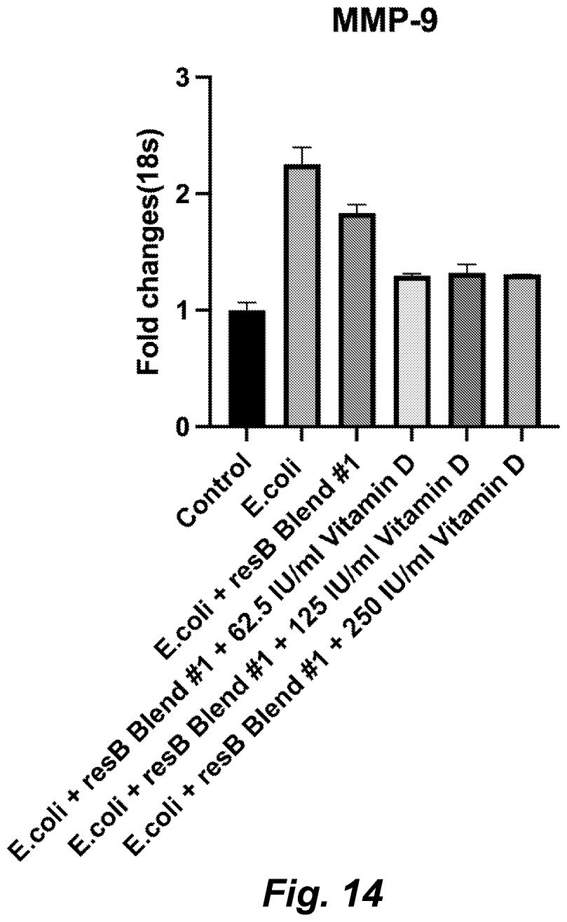

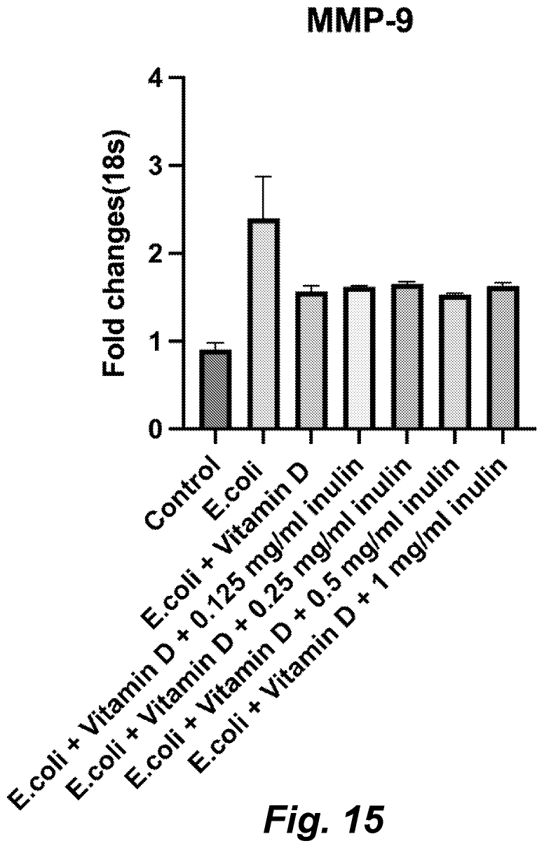

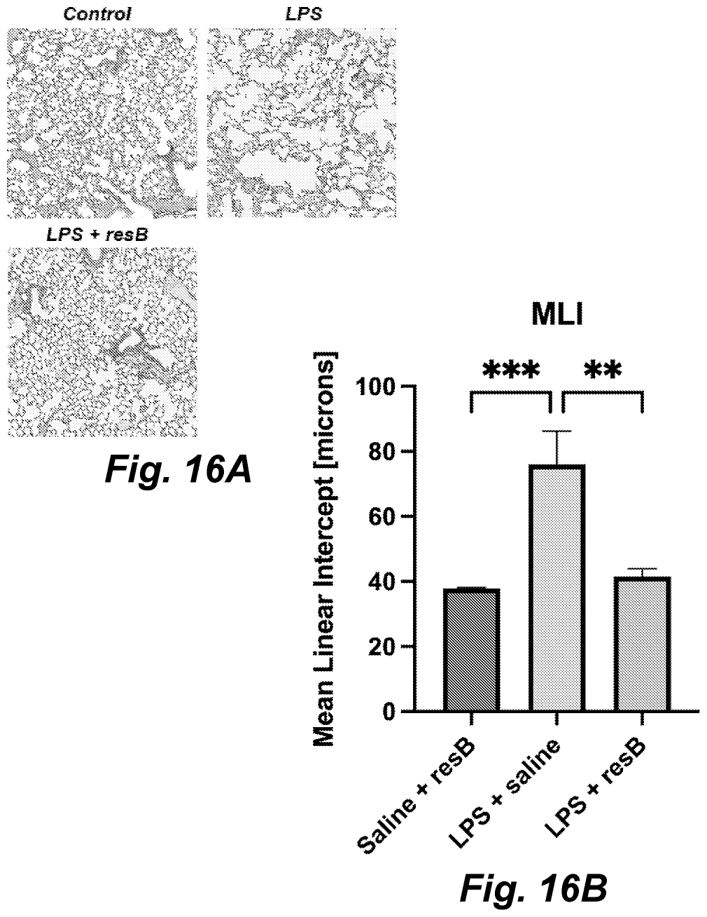

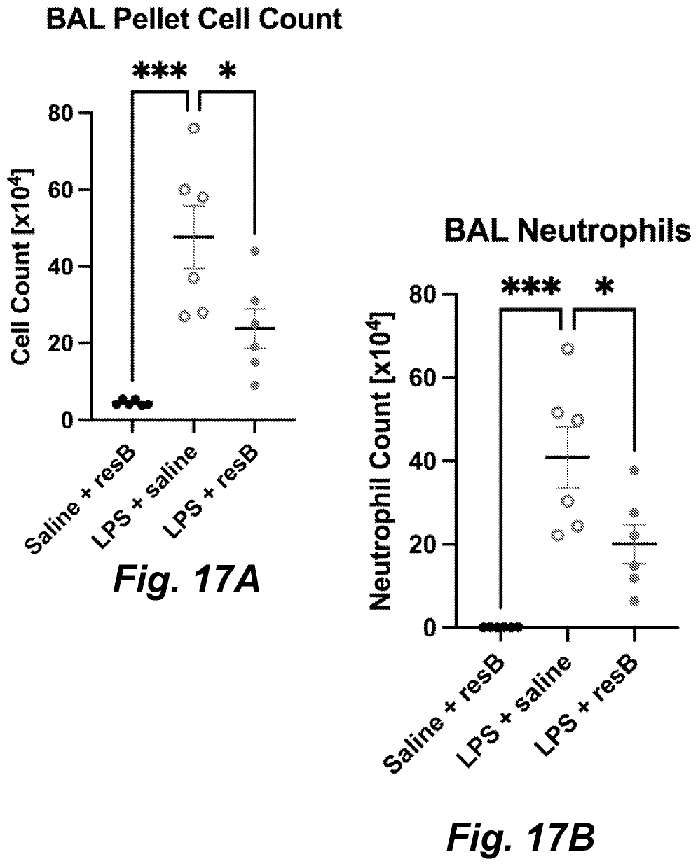

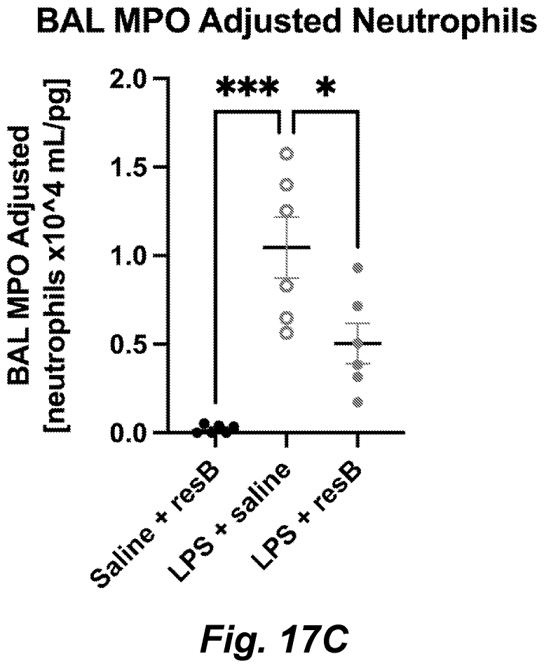

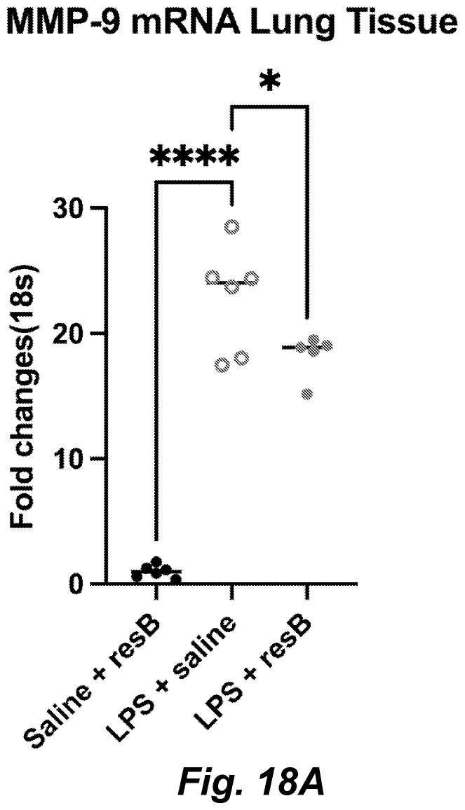

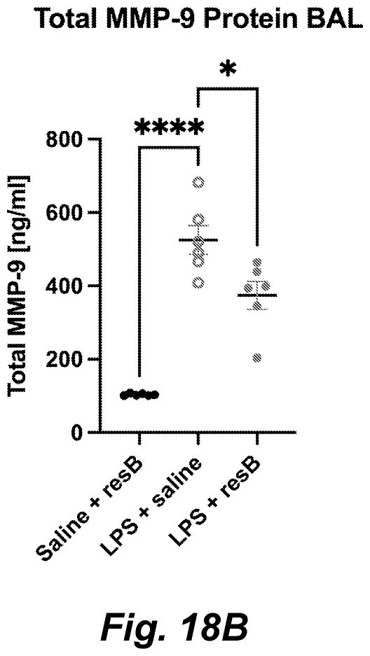

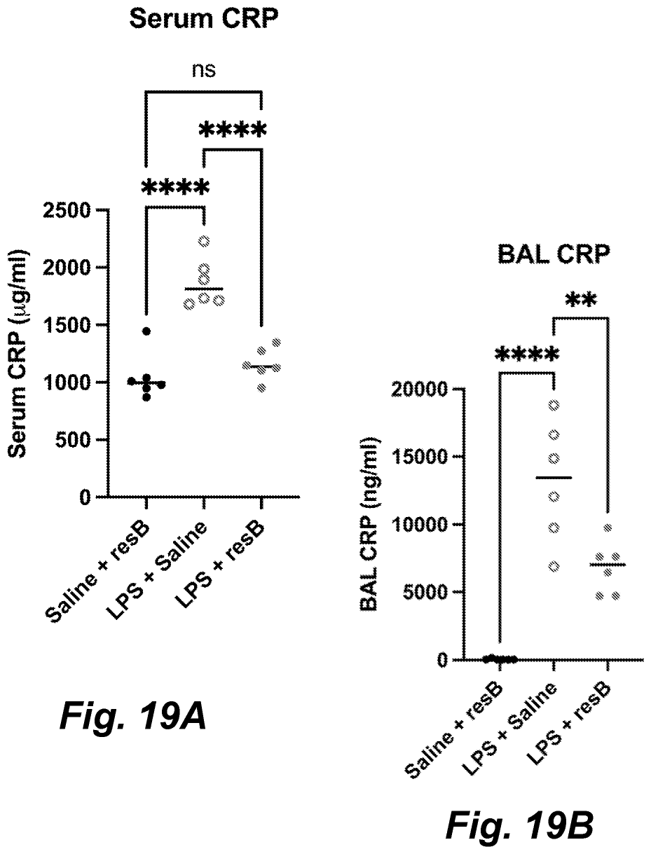

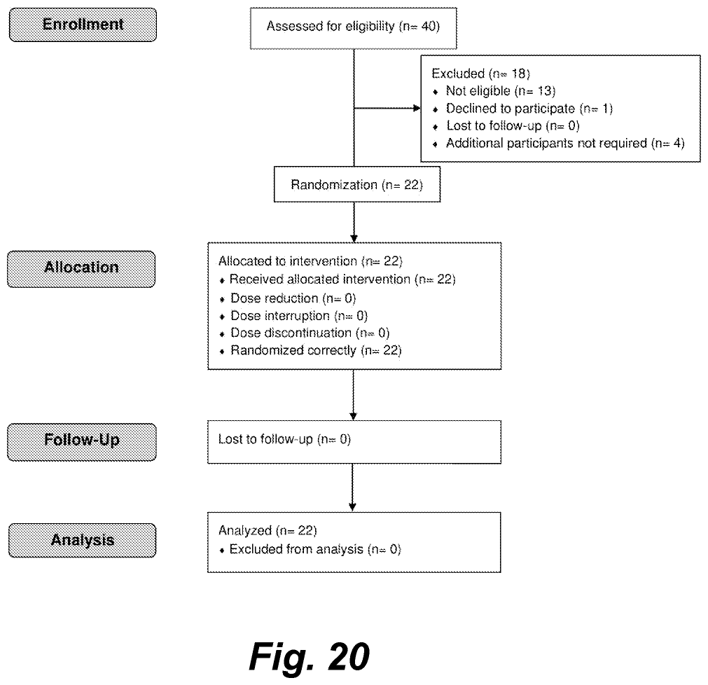

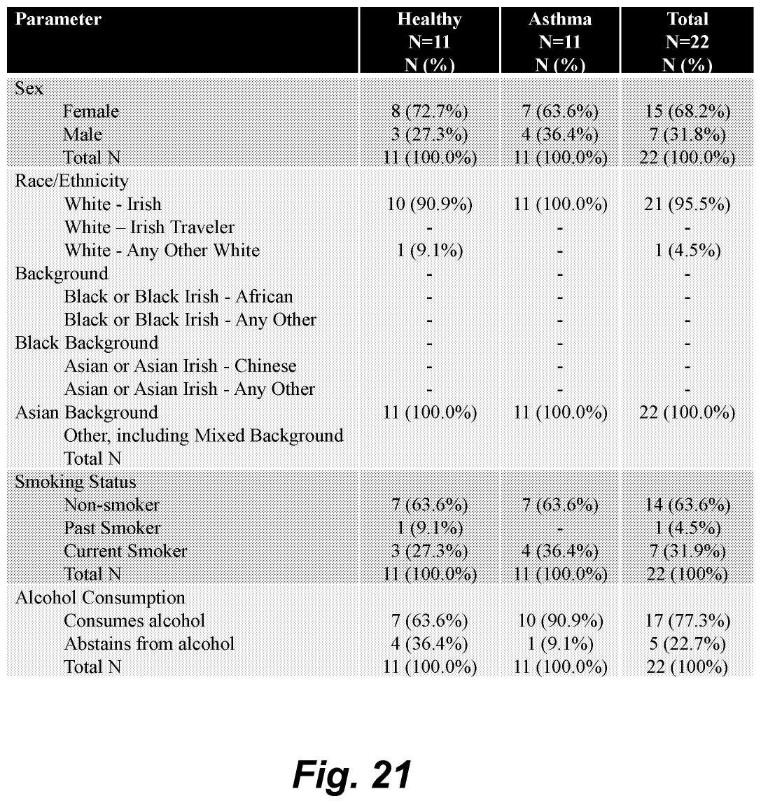

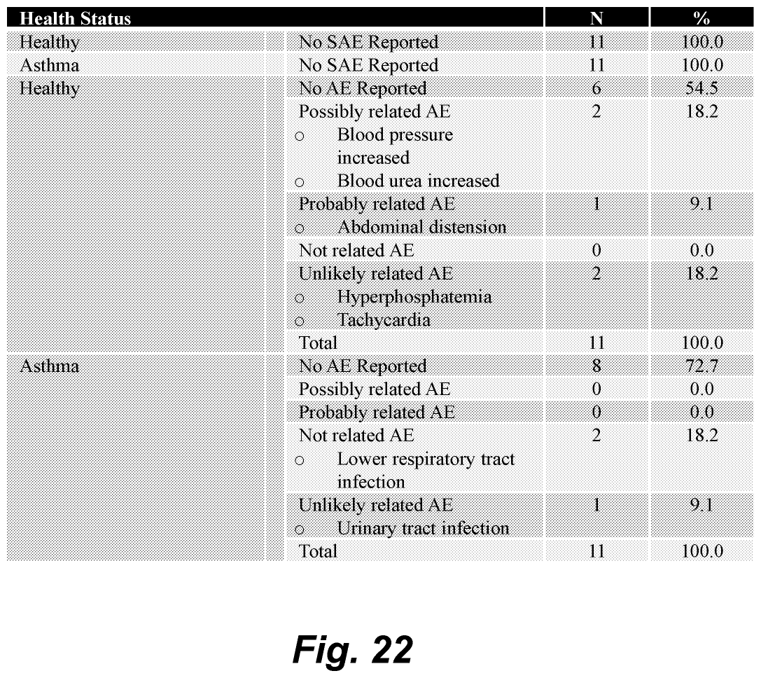

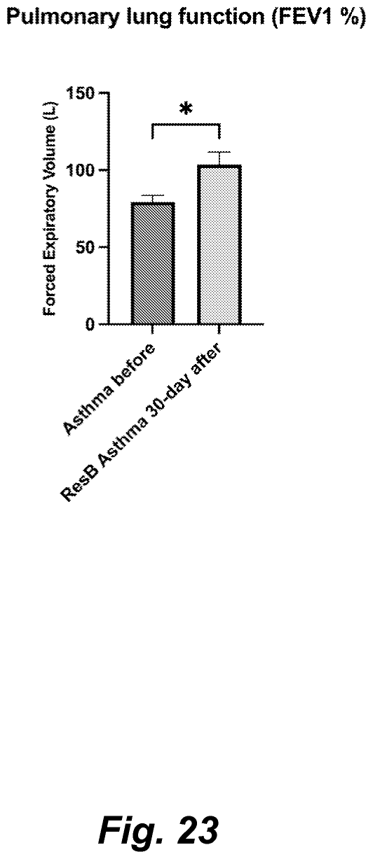

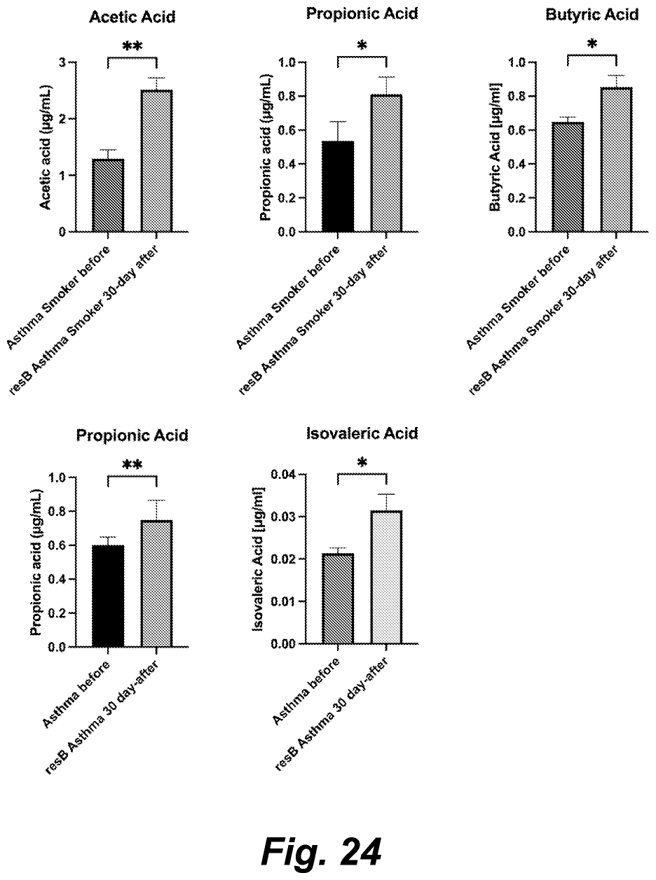

It has now been discovered that airways are not sterile even at birth but are occupied by a diverse microbiome, and that the airway microbiome, a population of pathogenic and commensal bacteria, is imbalanced (dysbiosis) during the development of lung disease. The study of the microbiome is a newly developing field, but less has been studied about the lung microbiome. It has been established that the tripeptide N-acetyl-proline-glycine-proline (Ac-PGP) derived from ECM breakdown plays a critical role in various adult chronic lung diseases by enhancing neutrophilic inflammation and endothelial permeability. Bacteria drive Ac-PGP release by initiating lung injury and ECM breakdown. It has now been shown that the inflammatory cascade, as shown in FIG. 1, is inhibited or prevented by Lactobacillus. It is contemplated, therefore, that probiotic-Lactobacillus spp. reduce the inflammation of lungs in various lung diseases by reduction of neutrophilic inflammation.

The pulmonary microbiome is well-established in both infants and adults. Dysbiosis of the pulmonary microbiome is a signature of respiratory diseases such as COPD, BPD, asthma, and CF. Data shows that harmful neutrophilic inflammation can result from dysbiosis in the lungs. Studies in human bronchial epithelial (HBE) cells, mouse models, and human BPD patients show the direct influence of gamma-proteobacteria in generating neutrophilic inflammation through increased levels of MMP-9, PE, and Ac-PGP. The relationship between lung dysbiosis and lung disease is established (Lal & Ambalavanan (2017) Early Hum. Dev. 105:35-39).

The gut microbiome, which has been the focus of study for a longer period, has a substantial relationship with the lung microbiome. Decreased populations of Lachnospira, Veillonella, Faecalibacterium, and Rothia are observed in children at risk for asthma in their first 100 days of life. COPD and asthma patients often have concurrent gastrointestinal symptoms of comparable severity. Oral administration of L. rhamnosus reduced nosocomial respiratory infections in children. Interactions between the two environments can occur through immune responses to bacterial byproducts, physical exchange through aspiration, and communication through metabolic byproducts.

Lactobacillus bacterial species are known for their commensal role in signaling beyond the gut. M'Rabet et al., (2005) showed that lactic acid-producing bacteria broadly have been established for their beneficial effects on airway narrowing in the treatment of lung disease as measured by the enhanced pause value. Lactobacillus rhamnosus in combination with ginger and Vitamin D has been shown to impact musculoskeletal disease and injuries through the synergy of the anti-inflammatory properties (Heck et al., 2018).

Gut microbiota influences both the gut immune system and the lung immune system via local or long-reaching interactions through CD8+ T cell, Th17, IL-25, IL-13, prostaglandin E2, and/or NF-κB-dependent pathways. The lung microbiota impacts the mucosal immunity and contributes to immune tolerance through neutrophil recruitment, production of pro-inflammatory cytokines mediated by receptor 2 (TLR2), and the release of antimicrobial peptides, such as β-defensin 2 stimulated by T helper 17 (Th17) cells.

Data indicates that probiotic Lactobacillus species inhibit inflammation in in vitro and animal models of chronic lung diseases. Accordingly, the present disclosure encompasses embodiments of ingestible probiotic dietary supplements that can lead to a reduction in bronchopulmonary pathologies such as BPD, COPD, CF, and ARDS. In addition to the probiotic bacteria, the nutraceutical compositions of the disclosure further comprise extracts of certain herbal plant species that can lead to an improvement in the respiratory health of the recipient subject. The probiotic therapy, therefore, include a combination of various Lactobacillus species such as Lactobacillus plantarum, Lactobacillus acidophilus and Lactobacillus rhamnosus to form a probiotic mixture. Formulations of the disclosure, one with only live bacteria and others with at least one heat-inactivated bacteria are contemplated as embodiments of the disclosure.

Herbal extracts have been shown to reduce symptoms of respiratory diseases. Most particularly, holy basil, turmeric, and vasaka can lessen symptoms and improve lung health in the short term while probiotics work to restore biosis and stop disease progression in the long term.

Herbal extracts can provide relief from symptoms of respiratory inflammation and disease. Holy basil, turmeric, and vasaka are plants used traditionally in Eastern medicinal practices with scientifically and anthropologically documented anti-inflammatory uses. Holy basil is known for its antioxidant, antimicrobial, and anti-inflammatory properties. Similarly, turmeric has a record of anti-cancer, antioxidant, anti-inflammatory, and free radical scavenging abilities. Vasaka is specifically known to assist proper respiratory function and decrease inflammation by downregulating TNF-alpha and IL-6. Data shows no impact of these three compounds on the growth of RSB11.RTM, RSB12.RTM, RSB13.RTM as measured by the number of colonies per plate. The addition of herbal extracts to a probiotic supplement blend can help mitigate symptoms of bronchopulmonary disease in conjunction with the well-characterized anti-inflammatory mechanisms of Lactobacillus probiotics.

Holy basil, also known as tulsi, is a medicinal aromatic herb from the family Lamiaceae that is indigenous to the Indian subcontinent (Jamshidi & Cohen (2017) Evid. Based Complement Alternat. Med. 2017:9217567). Studies in animal models have shown that the leaf extract of holy basil possesses anti-convulsant and anxiolytic activities (Okoli et al., (2010) Pharmacognosy Res. 2:36-40; Okomolo et al., (2011) Afr. J. Tradit. Complement. Altern. Med. 8:181-190). Holy basil aqueous leaf extract has also shown a promising effect on improving the immune response in bovine models (Mukherjee et al. 2005). In vitro and animal studies attest to holy basil leaf having potent pharmacological actions including adaptogenic (Jothie et al., (2016) Phytother. Res. 30:805-814), immunomodulatory (Sutili et al., (2016) J. Anim. Physiol. Anim. Nutr. (Berl) 100:1113-1120; Godhwani et al., (1988) J. Ethnopharmacol. 24:193-198), anticancer (Lin et al., (2014) Am. J. Chin. Med. 42:743-767), anti-inflammatory (Godhwani et al., (1987) J. Ethnopharmacol. 21:153-163; Kelm et al., (2000) Phytomed. 7:7-13), antioxidant (Kelm et al., (2000) Phytomed. 7:7-13), and antimicrobial (Nakamura et al., (1999) Mem. Inst. Oswaldo Cruz 94:675-678) effects.

Turmeric is a common flavoring and traditional medicinal agent (Esatbeyoglu et al., (2012) Angew Chem. Int. Ed. Engl. 51):5308-5332; Heger et al., (2014) Pharmacol. Rev. 66:222-307). Curcumin, a polyphenol derived from the plant, is reduced to several metabolites responsible for a diverse set of biological and pharmacological activities. In several studies, curcumin has shown a range of pharmacological effects including anti-cancer, anti-oxidation, anti-inflammatory, anti-bacterial, and free radical scavenging (Lim et al., (2001) J. Neurosci. 21:8370-8377).

Vasaka is a well-known plant in Ayurvedic and Unani medicine that has been used particularly for the treatment of respiratory tract ailments. Its active alkaloid compounds include vasicine, vasicinone and other derivatives (Gantait & Panigrahi (2018) J. Genet. Eng. Biotechnol. 16:545-552). Vasicine and vasicinone show bronchodilatory activity in vitro and in vivo (Cambridge et al., (1962) Nature 196:1217). The alkaloid fraction from vasaka has anti-inflammatory properties and decreases TNF-α and IL-6 (Amala & Sujatha (2021) Bioimpacts 11:15-22). Vasaka improves respiratory function and has antitussive properties (Narimanian et al., (2005) Phytomed. 12:539-547; Dhuley J. N. (1999) J. Ethnopharmacol. 67:361-365).

The combination of herbal extracts and a probiotic supplement according to the disclosure and formulated as a dietary supplement helps mitigate symptoms of bronchopulmonary disease in conjunction with the anti-inflammatory mechanisms of the probiotics. The present disclosure, therefore, encompasses embodiments of a probiotic pharmaceutical composition that delivers 3 strains of Lactobacillus (RSB11.RTM, RSB12.RTM, RSB13.RTM) to the gut lining of human subjects to reduce neutrophilic inflammation as a result of bronchopulmonary disease. At least one of the three strains of Lactobacillus should be a population of live organisms capable of proliferation in the recipient subject. A non-living strain of Lactobacillus that is included in the combination of bacterial strains can be, but is not limited to, a heat-killed non-proliferative strain.

In addition to the three Lactobacillus strains, the compositions can include herbal extracts from holy basil, turmeric, and vasaka for respiratory relief. The compositions of this present disclosure are therefore man-made compositions without any naturally occurring counterparts.

Accordingly, one aspect of the disclosure encompasses embodiments of a food product, dietary supplement, medical food, or nutraceutical, wherein the food product, dietary supplement, medical food, or nutraceutical can comprise at least viable one probiotic bacterial strain selected from Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, the food product, dietary supplement, medical food, or nutraceutical can comprise a mix of two or three of the probiotic bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, the food product, dietary supplement, medical food, or nutraceutical can comprise a mix of the bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, the food product, dietary supplement, medical food, or nutraceutical can consist of a mix of the bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal can be selected from the group consisting of holy basil, turmeric, and vasaka.

In some embodiments of this aspect of the disclosure, at least one Lactobacillus species can be heat-killed.

In some embodiments of this aspect of the disclosure, the percent weights (% wt) of the Lactobacillus plantarum RSB11. RTM, Lactobacillus acidophilus RSB12.RTM, and Lactobacillus rhamnosus RSB13.RTM in the bacterial mix can be, respectively, for RSB11.RTM: 1%-30%; 5%-30%; 5%-20%; 5%-15%; 5%-10%; 10%-30%; 10%-20%; 10%-15%; 15%-30%; 20%-30%, for RSB12.RTM: 1%-30%; 5%-30%; 5%-20%; 5%-15%; 5%-10%; 10%-30%; 10%-20%; 10%-15%; 15%-30%; 20%-30%, and for RSB13.RTM: 1%-30%; 5%-30%; 5%-20%; 5%-15%; 5%-10%; 10%-30%; 10%-20%; 10%-15%; 15%-30%; 20%-30%, with the total percent weights adjusted to 100% wt with at least one excipient, and wherein the ratio of bacteria mix:excipient can be about 10:about 1, about 7:about 1, about 5:about 1, about 4:about 1, about 3:about 1, about 2:about 1, about 1:about 1, about 1:about 2, about 1:about 3, about 1:about 4, about 1:about5, about 1:about7, about 1:about 10.

In some embodiments of this aspect of the disclosure, the percent weights (% wt) of the herbs, or extracts thereof in the herbal mix can be, respectively, turmeric extract: 1%-30%; 5%-30%; 5%-20%; 5%-15%; 5%-10%; 10%-30%; 10%-20%; 10%-15%; 15%-30%; 20%-30%, holy basil extract: 1%-30%; 5%-30%; 5%-20%; 5%-15%; 5%-10%; 10%-30%; 10%-20%; 10%-15%; 15%-30%; 20%-30%, and for vasaka extract: 1%-30%; 5%-30%; 5%-20%; 5%-15%; 5%-10%; 10%-30%; 10%-20%; 10%-15%; 15%-30%; 20%-30% with the total percent weights adjusted to 100% wt with at least one excipient.

In some embodiments of this aspect of the disclosure, the food product, dietary supplement, medical food, or nutraceutical the bacterial mix can consist of Lactobacillus plantarum RSB11.RTM, Lactobacillus acidophilus RSB12.RTM, and Lactobacillus rhamnosus RSB13.RTM, and an excipient consisting of magnesium stearate; silica, and MCC and the herbal mix can consist of turmeric extract, holy basil extract, and vasaka extract.

In some embodiments of this aspect of the disclosure, the food product, dietary supplement, medical food, or nutraceutical can be formulated for oral consumption.

In some embodiments of this aspect of the disclosure, the food product, dietary supplement, medical food, or nutraceutical can be one or more nutritionally acceptable carriers, one or more nutritionally acceptable diluents, one or more nutritionally acceptable excipients, one or more nutritionally acceptable adjuvant or nutritionally active ingredient, or any combination thereof, in addition to the combination of the bacteria and the at least one herb, or extract thereof.

In embodiments of this aspect of the disclosure, the nutraceutical can further comprise at least one nutritionally active ingredient.

In embodiments of this aspect of the disclosure, the at least one nutritionally active ingredient can be a vitamin, a micronutrient, a mineral, a prebiotic fiber, a fatty acid, an amino acid, or any combination thereof.

In embodiments of this aspect of the disclosure, the at least one vitamin can be ascorbic acid (vitamin C), D, E, A, E, K, thiamine (B1), riboflavin (B2), niacin (B3), pantothenic acid (B5), B6, B7, B9, B12, or any combination thereof and the at least one mineral can be sulfur, potassium, chlorine, sodium, calcium, phosphorus, magnesium, iron, zinc, manganese, copper, iodine, chromium, molybdenum, selenium, cobalt, or any combination thereof.

In embodiments of this aspect of the disclosure, the food product, dietary supplement, medical food, or nutraceutical can be formulated for oral consumption.

In embodiments of this aspect of the disclosure, the food product, dietary supplement, medical food, or nutraceutical can further comprising a nutritionally acceptable carrier, nutritionally acceptable diluent, nutritionally acceptable excipient, nutritionally acceptable adjuvant or nutritionally active ingredient in addition to the bacteria and the at least one herb or herb extract.

In some embodiments of this aspect of the disclosure, the food product, dietary supplement, medical food, or nutraceutical can be an orally ingestible form selected from the group consisting of a capsule, microcapsule, tablet, granule, powder, troche, pill, suspension, and syrup.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

Constituent of Nutraceutical- resB
resB blend 1 (RSB11.RTM + RSB12.RTM + RSB13.RTM)
Vasaka Extract^{a}
Organic Holy Basil Extract^{b}
Turmeric Extract^{c}
Excipient-MCC
resB blend 1
RSB11.RTM
RSB12.RTM
RSB13.RTM
Excipient- MCC
^{a}vasaka extract (Adhatoda casica, leaves) supplied by Karallief, Cambridge, MA, USA
^{b}holy basil extract (Ocimum sanctum, leaves) supplied by Star Hi Herbs, Kamataka, India
^{c}turmeric extract (Curcuma longa, root) supplied by Naturex, France

### Example 2

| Constituent of Nutraceutical-resB | mg |
|---|---|
| resB blend 1 (RSB11.RTM + RSB12.RTM + RSB13.RTM) | 100.00 |
| Turmeric Extract | 30.45 |
| Organic Holy Basil Extract | 42.63 |
| Vasaka Extract | 48.72 |
| Excipient-MCC | 28.21 |
| resB blend 1 | % |
| Lactobacillus plantarum, RSB11.RTM | 16.2 |
| Lactobacillus acidophilus, RSB12.RTM | 31.4 |
| Lactobacillus rhamnosus, RSB13.RTM | 14.2 |
| Excipient-MCC | 38 |

### Example 3

| Constituent of | B CFU | |
|---|---|---|
| Nutraceutical-resB.RTM | (label) | mg |
| resB.RTM blend 1 (RSB11.RTM + | 15 | 125.0 |
| RSB12.RTM + RSB13.RTM) | | |
| Magnesium Stearate | | 8.0 |
| Silica | | 8.0 |
| MCC | | 91.2 |
| SUM- Active mass | | 232.2 |
| Turmeric Extract | | 30.0 |
| Organic Holy Basil Extract | | 42.0 |
| Vasaka Extract | | 48.0 |
| SUM- Extracts | | 120.0 |
| Capsule Fill | | 352.2 |

| resB blend 1 | B CFU | Mass (mg) | % w/w |
|---|---|---|---|
| RSB11.RTM | 7.875 | 20.25 | 16.2 |
| RSB12.RTM | 8.25 | 39.00 | 31.2 |
| RSB13.RTM | 6.375 | 17.75 | 14.2 |
| MCC | | 48.00 | 38.4 |
| SUM | 22.5 | 125.00 | 100.0 |

### Example 4

### Herbal Extracts

1. Vasaka Extract from Leaves of Adathoda vasica Plant Part: Leaves

### Vasaka Extraction Per Manufacturer (Karalieff, Inc, Cambridge, MA, U.S.A.):

Vasaka leaves are charged to the extractor along with ethanol and water. The extraction is by heating the mass in a closed system by repumping the extract to the herb bed. This process is repeated and filtered through polypropylene filter bags of 5 micron size. The extracts are concentrated under reduced pressure at low temperature. This is charged to a drier unit to dry and separate the product in a powder form. This is further powdered in a multimill to a fine mesh size. It is sieved to a uniform particle size. The product is mixed in a blender along with dextrin to make a uniform and homogenous batch. Finally the product is heat sterilized 100° C. to 105° C. for 2 hrs and sieved before packaging. Final product contains about 20% by weight of saponins.

2. Turmeric (Curcuma longa L.) Root Containing Curcuminoids

### Turmeric Extraction Per Manufacturer (Naturea, Inc, South Hackensack, NJ, U.S.A.):

Extraction svent: acetone 100%, then ethanol 100%; Extract ratio: 25:1

Composition: Curcuminoid: >95% by HPLC; Curcumin: 70.00-80.00% by HPLC;

Bisdemethoxycurcumin: 2.50-6.50% by HPLC; Demethoxycurcumin: 15.00-25.00% by HPLC; moisture content: <2%; particle size: 40 msh.

3. Holy Basil Extract from Dry Leaves of Ocimum sanctum

### Holy Basil Extraction Per Manufacturer (STAR Hi Herbs Pvt Ltd., Bangalore, India):

Final extract Ratio: 10:1 Solvent: Methanol

Chemical analysis: Ursolic acid and Oleanolic acid: Not less than 2.5% w/w By HPLC.

### Example 5

### Percent Weiqhts (% Wt) Ranges for Bacterial Strains

RSB11.RTM: 1%-30%; 5%-30%; 5%-20%; 5%-15%; 5%-10%; 10%-30%; 10%-20%; 10%-15%; 15%-30%; 20%-30%
RSB12.RTM: 1%-30%; 5%-30%; 5%-20%; 5%-15%; 5%-10%; 10%-30%; 10%-20%; 10%-15%; 15%-30%; 20%-30%
RSB13.RTM: 1%-30%; 5%-30%; 5%-20%; 5%-15%; 5%-10%; 10%-30%; 10%-20%; 10%-15%; 15%-30%; 20%-30% MCC: to 100% wt

### Exemplary Ratios of Bacteria Mix:Excipient (Such as MCC)

about 10:about 1, about 7:about 1, about 5:about 1, about 4:about 1, about 3:about 1, about 2:about 1, about 1:about 1, about 1:about 2, about 1:about 3, about 1:about 4, about 1:about 5, about 1:about 7, about 1:about 10.

### Example 6

### Percent Weiqhts (% Wt) Ranges for Nutraceuticals in Nutraceutical Mix

Turmeric Extract: 1%-30%; 5%-30%; 5%-20%; 5%-15%; 5%-10%; 10%-30%; 10%-20%; 10%-15%; 15%-30%; 20%-30%
Organic Holy Basil Extract: 1%-30%; 5%-30%; 5%-20%; 5%-15%; 5%-10%; 10%-30%; 10%-20%; 10%-15%; 15%-30%; 20%-30%
Vasaka Extract: 1%-30%; 5%-30%; 5%-20%; 5%-15%; 5%-10%; 10%-30%; 10%-20%; 10%-15%; 15%-30%; 20%-30%
MCC: to 100% wt

### Exemplary Ratios of Bacteria Mix:Nutraceutical Mix

about 10:about 1, about 7:about 1, about 5:about 1, about 4:about 1, about 3:about 1, about 2:about 1, about 1:about 1, about 1:about 2, about 1:about 3, about 1:about 4, about 1:about 5, about 1:about 7, about 1:about 10

What is claimed:

1. A food product, dietary supplement, medical food, or nutraceutical, wherein the food product, dietary supplement, medical food, or nutraceutical comprises at least viable one probiotic bacterial strain selected from Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal is selected from the group consisting of holy basil, turmeric, and vasaka.

2. The food product, dietary supplement, medical food, or nutraceutical of claim 1, comprising a mix of two or three of the probiotic bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal is selected from the group consisting of holy basil, turmeric, and vasaka.

3. The food product, dietary supplement, medical food, or nutraceutical of claim 1, comprising a mix of the bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal is selected from the group consisting of holy basil, turmeric, and vasaka.

4. The food product, dietary supplement, medical food, or nutraceutical of claim 1, consisting of a mix of the bacterial strains Lactobacillus plantarum RSB11.RTM (ATCC Patent Deposit Number PTA-127171), Lactobacillus acidophilus RSB12.RTM (ATCC Patent Deposit Number PTA-127172), and Lactobacillus rhamnosus RSB13.RTM (ATCC Patent Deposit Number PTA-127173), and a herbal mix comprising at least one herbal, or an extract thereof, wherein the at least one herbal is selected from the group consisting of holy basil, turmeric, and vasaka.

5. The dietary supplement claim 2, in which at least one Lactobacillus species is heat-killed.

6. The food product, dietary supplement, medical food, or nutraceutical of claim 3, wherein the percent weights (% wt) of the Lactobacillus plantarum RSB11.RTM, Lactobacillus acidophilus RSB12.RTM, and Lactobacillus rhamnosus RSB13.RTM in the bacterial mix are, respectively, for RSB11.RTM: 1%-30%; 5%-30%; 5%-20%; 5%-15%; 5%-10%; 10%-30%; 10%-20%; 10%-15%; 15%-30%; 20%-30%, for RSB12.RTM: 1%-30%; 5%-30%; 5%-20%; 5%-15%; 5%-10%; 10%-30%; 10%-20%; 10%-15%; 15%-30%; 20%-30%, and for RSB13.RTM: 1%-30%; 5%-30%; 5%-20%; 5%-15%; 5%-10%; 10%-30%; 10%-20%; 10%-15%; 15%-30%; 20%-30%, with the total percent weights adjusted to 100% wt with at least one excipient, and wherein the ratio of bacteria mix:excipient is about 10:about 1, about 7:about 1, about 5:about 1, about 4:about 1, about 3:about 1, about 2:about 1, about 1:about 1, about 1:about 2, about 1:about 3, about 1:about4, about 1:about5, about 1:about7, about 1:about 10.

7. The food product, dietary supplement, medical food, or nutraceutical of claim 1, wherein the percent weights (% wt) of the herbs, or extracts thereof in the herbal mix are, respectively, turmeric extract: 1%-30%; 5%-30%; 5%-20%; 5%-15%; 5%-10%; 10%-30%; 10%-20%; 10%-15%; 15%-30%; 20%-30%, holy basil extract: 1%-30%; 5%-30%; 5%-20%; 5%-15%; 5%-10%; 10%-30%; 10%-20%; 10%-15%; 15%-30%; 20%-30%, and for vasaka extract: 1%-30%; 5%-30%; 5%-20%; 5%-15%; 5%-10%; 10%-30%; 10%-20%; 10%-15%; 15%-30%; 20%-30% with the total percent weights adjusted to 100% wt with at least one excipient.

8. The food product, dietary supplement, medical food, or nutraceutical of claim 3, wherein (i) the bacterial mix consists of Lactobacillus plantarum RSB11.RTM, Lactobacillus acidophilus RSB12.RTM, and Lactobacillus rhamnosus RSB13.RTM, and an excipient consisting of magnesium stearate; silica, and MCC and (ii) the herbal mix consists of turmeric extract, holy basil extract, and vasaka extract.

9. The food product, dietary supplement, medical food, or nutraceutical of claim 6, further comprising a nutritionally acceptable carrier, nutritionally acceptable diluent, nutritionally acceptable excipient, nutritionally acceptable adjuvant or nutritionally active ingredient in addition to the bacteria and the at least one herb or herb extract.

10. The food product, dietary supplement, medical food, or nutraceutical of claim 3 comprises at least one nutritionally active ingredient.

11. The food product, dietary supplement, medical food, or nutraceutical of claim 10, wherein the at least one nutritionally active ingredient is a vitamin, a micronutrient, a mineral, a prebiotic fiber, a fatty acid, an amino acid, or any combination thereof.

12. The food product, dietary supplement, medical food, or nutraceutical of claim 11, wherein the at least one vitamin is ascorbic acid (vitamin C), D, E, A, E, K, thiamine (B1), riboflavin (B2), niacin (B3), pantothenic acid (B5), B6, B7, B9, B12, or any combination thereof and the at least one mineral is sulfur, potassium, chlorine, sodium, calcium, phosphorus, magnesium, iron, zinc, manganese, copper, iodine, chromium, molybdenum, selenium, cobalt, or any combination thereof.

13. The food product, dietary supplement, medical food, or nutraceutical claim 1 formulated for oral consumption.

14. The food product, dietary supplement, medical food, or nutraceutical of claim 9, wherein the food product, dietary supplement, medical food or nutraceutical is an orally ingestible form selected from the group consisting of a capsule, microcapsule, tablet, granule, powder, troche, pill, suspension, and syrup.
